(19) United States
(12) Patent Application Publication
(10) Pub. No.: US 2001/0028886 A1
(43) Pub. Date: Oct. 11, 2001

(54) Solid plaster-based cosmetic composition comprising a specific setting-kinetics modifier, preparation process and uses thereof

(76) Inventors: Valerie Robert, Paris (FR); Patricia Lemann, Creteil (FR); Annick Collette, Saint-Maur des Fosses (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/736,303

(22) Filed: Dec. 15, 2000

(30) Foreign Application Priority Data
Dec. 15, 1999 (FR) 99 15818

(51) Int. Cl. A61K 7/02; A61K 9/70

(52) U.S. Cl. 424/401; 424/443

(57) ABSTRACT

Plaster-based cosmetic compositions comprising at least one setting-kinetics modifier chosen from calcium monophosphates, formaldehyde/4-aminobutyric acid/calcium salt condensates, and starch-coated gypsums, which can be used, for example, in the field of cosmetics. The compositions may be solid compositions. Processes for preparing plaster based compositions, for example cosmetic compositions, comprising a) preparing a pulverulent mixture comprising at least calcium sulphate hemihydrate in powder form, b) adding an aqueous phase to said pulverulent mixture, c) blending said pulverulent mixture and said aqueous phase to obtain a castable mixture, d) pouring said castable mixture into a mold,

e) leaving said castable mixture in said mold to harden by hydrating said calcium sulphate hemihydrate to calcium sulphate dehydrate, and f) introducing at least one setting-kinetics modifier. Compositions, for example cosmetic compositions, which can be obtained by the process.

The present invention relates to solid plaster-based cosmetic compositions which can be used, for example, in the field of make-up. The invention also relates to processes for manufacturing solid compositions using plaster and compositions, for example a cosmetic composition, obtained by this process.

It is known practice to manufacture cosmetic compositions in solid form using plaster. These cosmetic compositions may be, for example, eyeshadows, blushers, face powders or body powders. They can be in the form of sticks, pencils or cakes. The use of plaster to manufacture these solid compositions is disclosed, for example, in U.S. Pat. No. 4,724,138. According to the known processes, a pulverulent phase containing plaster (calcium sulphate hemihydrate) is mixed with an aqueous phase to give a paste which is shaped by molding, the hydration reaction of the plaster into calcium sulphate dihydrate bringing about solidification of the composition. The use of plaster as a solidifying agent is advantageous since it makes it possible to replace the compacting operation, which is usually needed to obtain a composition in solid form from pulverulent products, with a molding operation, which is easier and less expensive to carry out and allows more diverse forms to be obtained.

The inventors have found that these compositions were still not entirely satisfactory in regards to the cohesion and strength of the final product: for example, the compositions can adhere to their packaging article and they can remain attached to this packaging article. The compositions then show spaces or holes and their external appearance is no longer uniform. The finished product lacks strength. It may thus turn out that on opening the product, some of the composition becomes detached and is lost to the consumer. This phenomenon may be observed when the composition comprises, for example, a high content of large particles or when it comprises various fillers whose particles are very varied in shape.

An aim of the present invention is to overcome at least some of these drawbacks.

The inventors have discovered, surprisingly and unexpectedly, that by adding a specific compound, namely a setting-kinetics modifier, to these compositions, compositions with desirable properties such as a smooth and uniform outer appearance can be prepared.

As explained above, to prepare a plaster-based composition, a pulverulent mixture comprising calcium sulphate hemihydrate is conventionally prepared and is then mixed with water to convert this calcium sulphate hemihydrate into calcium sulphate dihydrate, thus resulting in a solid product. The time taken for the “pulverulent mixture +water” paste to become solid is known as the setting time.

It is known practice in the plaster-making industry to use setting-time modifiers in plaster-based compositions, for example for reasons of industrial constraints such as existence of a target setting time, time required for the casting operations, etc.

However, no disclosure has ever been given of a specific setting-kinetics modifier which can allow the preparation of a plaster-based cosmetic composition with a particularly smooth and uniform surface.

It has been observed, for example, that setting-kinetics modifiers such as calcium monophosphates, formaldehyde/4-aminobutyric acid/calcium salt condensates, optionally dispersed in calcium sulphate, starch-coated gypsums and mixtures of any of the foregoing give compositions which have a particularly homogeneous surface.

One subject of the present invention is the use of at least one setting-kinetics modifier chosen from calcium monophosphates, formaldehyde/4-aminobutyric acid/calcium salt condensates, optionally dispersed in calcium sulphate, starch-coated gypsums and mixtures of any of the foregoing, in or for the preparation of a solid cosmetic composition comprising calcium sulphate dihydrate, with the aim of giving said composition a smooth and uniform surface.

One subject of the present invention is thus also a solid cosmetic composition comprising calcium sulphate dihydrate, further comprising at least one setting-kinetics modifier chosen from calcium monophosphates, formaldehyde/4-aminobutyric acid/calcium salt condensates, optionally dispersed in calcium sulphate, starch-coated gypsums and mixtures of any of the foregoing, and a cosmetically and physiologically acceptable medium.

One subject of the invention is also a process for preparing a solid plaster-based cosmetic composition, comprising:

a) preparing a pulverulent mixture comprising at least calcium sulphate hemihydrate in powder form;

b) adding an aqueous phase to the pulverulent mixture;

c) blending the pulverulent mixture and the aqueous phase to obtain a castable mixture;

d) pouring the castable mixture into a mold;

e) leaving the mixture to harden by hydrating the calcium sulphate hemihydrate to calcium sulphate dehydrate; and

f) introducing at least one setting-kinetics modifier chosen from calcium monophosphates, formaldehyde/4-aminobutyric acid/calcium salt condensates, optionally dispersed in calcium sulphate, starch-coated gypsums and mixtures of any of the foregoing.

The invention also relates to a cosmetic composition which can be manufactured by the process according to the invention. The composition thus obtained is entirely suitable for make-up. It can be used as a blusher, an eyeshadow, a powder for the face and a powder for the body.

In one embodiment, the compositions obtained have desirable properties such as no spaces, coarseness or aggregates. In one embodiment, the compositions obtained do not adhere to their packaging, whatever it may be. In one embodiment, the compositions obtained show good cohesion, good strength and a particularly uniform and smooth outer appearance.

One subject of the present invention is also a non-therapeutic process for making up the skin, characterized in that a composition as defined above is applied to the skin.

Examples of setting-kinetics modifiers which can be used according to the invention include setting-time retarders, for example sodium citrate, calcium monophosphate and formaldehyde/4-aminobutyric acid/calcium salt condensates, optionally dispersed in calcium sulphate, setting-time accelerators, for example gypsum, starch-coated gypsum and sodium sulphates, and mixtures of any of the foregoing.

The setting-kinetics modifier can be chosen from calcium monophosphates, formaldehyde/4-aminobutyric acid/calcium salt condensates dispersed in calcium sulphate, starch-coated gypsum and mixtures of any of the foregoing.

Examples of commercially available setting-kinetics modifiers include calcium hydrogen phosphate monohydrate sold under the trade name “Retardateur RA 01” by the company Lafarge, the formaldehyde/4-aminobutyric acid/calcium salt condensate dispersed to a proportion of 11% in calcium sulphate sold under the trade name “Retardan GK” by Tricosal, and the starch-coated gypsum sold under the trade name “BMA” by Prestia Lafarge.

In one embodiment of the invention, the setting-kinetics modifier comprises a formaldehyde/4-aminobutyric acid/calcium salt condensate dispersed in calcium sulphate. This setting-kinetics modifier can also comprise a mixture of i) formaldehyde/4-aminobutyric acid/calcium salt condensate dispersed in calcium sulphate and ii) starch-coated gypsum. The formaldehyde/4-aminobutyric acid/calcium salt condensate can be dispersed to a proportion of 11% in calcium sulphate.

The setting-kinetics modifier according to the invention can comprise a calcium monophosphate.

The setting-kinetics modifier according to the invention can also comprise sodium citrate.

Introducing the setting-kinetics modifier according to the invention can be carried out at any point in the process for manufacturing the compositions according to the invention, for example during the preparation of the pulverulent mixture, or at the same time as adding the aqueous phase. The setting-kinetics modifier can be in the form of a pulverulent compound or dissolved in a solvent.

In one embodiment, the modifier according to the invention can be added to the pulverulent mixture during the preparation of the pulverulent mixture.

The setting-kinetics modifier is generally present in the pulverulent mixture at a concentration ranging from 0.001% to 5% by weight relative to the total weight of the pulverulent mixture, can range from 0.01% to 1% by weight, and can also range from 0.01% to 0.6% by weight.

It emerges from the process for manufacturing the compositions according to the invention that the weight content, relative to the total weight of the pulverulent mixture prepared in this process, of a specific pulverulent compound is substantially equal to the weight content, relative to the total weight of the final composition, of said specific pulverulent compound.

In one embodiment, the setting-kinetics modifier is generally present in the final compositions according to the invention in a concentration ranging from 0.001% to 5% by weight relative to the total weight of the final composition, can range from 0.01% to 1% by weight, and can also range from 0.01% to 0.6% by weight.

The calcium sulphate hemihydrate used according to the invention can be in its a form and in its β form. Calcium sulphate hemihydrate is a compound that is well known to those skilled in the art.

The amount of plaster (calcium sulphate hemihydrate) present in the pulverulent mixture generally ranges from 10% to 70% by weight relative to the total weight of the pulverulent mixture, can range from 15% to 35% by weight, and can also range from 20% to 30% by weight.

In one embodiment, the calcium sulphate dihydrate is present in the final composition in a concentration generally ranging from 10% to 70% by weight relative to the 5 weight of the final composition, and can also range from 15% to 35% by weight.

For the purposes of the present invention, the expression “cosmetically and physiologically acceptable medium” means a medium which is compatible with the skin, in other words any area of facial and body skin. In one embodiment, the composition according to the invention can also further comprise at least one hydrophilic pulverulent material.

The expression “hydrophilic pulverulent material” means both pulverulent materials that are hydrophilic by nature (not treated by coating or chemical grafting) and pulverulent materials treated by coating or chemical grafting so as to give them hydrophilic properties.

In order to determine, according to the invention, whether a pulverulent material is hydrophobic or hydrophilic, the test defined below is carried out. A test tube 20 mm in diameter is filled with 20 ml of water. 2 grams of powder are added to the tube without shaking and the behavior of the powder is observed for a maximum of 5 minutes. If the powder remains entirely at the surface, it is considered as “hydrophobic”. In the opposite case, it is considered as “hydrophilic”.

The naturally hydrophilic pulverulent materials may be chosen, for example, from:

micas, which are potassium aluminium silicates of varied composition, of natural origin, for example muscovite, phlogopite, lepidolite, biotite and sericite, and of synthetic origin,

bismuth oxychloride,

silicas, which may be in the form chosen form platelets and spheres, for example the silica sold under the name “Silica Beads SB 700” by the company Miyoshi,

hydrophilic polymer powders, which are of synthetic origin, for instance polyacrylates, for example the product sold under the name “Micropearl M 100” by the company Matsumoto, acrylic polyamides for example those sold by the company Oris, polyurethanes for example the product sold under the name “Plastic Powder D 800” by the company Toshnu, and cellulose derivatives and starch derivatives, for example porous cellulose microspheres,

kaolin, which is an aluminium silicate hydrate,

hydroxyapatite,

zinc oxide or titanium oxide, for their covering power for example, these products possibly being used in nanopigment form for their screening effect,

calcium carbonate,

magnesium carbonates and hydrocarbonates, which facilitate the binding of fragrances,

and mixtures of any of the foregoing.

The hydrophilic pulverulent materials can also be pulverulent materials which have been made hydrophilic by coating or chemical grafting with materials for example chitosan, titanium dioxide, silica and hydrophilic polymers, for example sulphonic polyesters, polyquaternary ammoniums, and mixtures of any of the foregoing.

In one embodiment, the hydrophilic pulverulent materials can be chosen from hydrophilic treated and coated talcs, hydrophilic treated and coated polyamide powders, hydrophilic treated and coated polyethylene powders, hydrophilic treated and coated powders of expanded copolymer of vinylidene chloride, acrylonitrile and methyl (meth)acrylate, hydrophilic treated and coated polyfluoro powders, hydrophilic treated and coated silicone powders, hydrophilic treated and coated acrylic copolymer powders, hydrophilic treated and coated polystyrene powders and hydrophilic treated and coated pigments, and mixtures of any of the foregoing.

Examples of hydrophilic pulverulent materials which may be mentioned include chitosan-coated talc sold by Daito under the name “Talc CT 2 MSA” and mica coated with silica microspheres, sold by Catalysts and Chemicals under the name “Cashmir B3”.

The hydrophilic pulverulent materials can also be chosen from the hydrophilic pigments and nacres and flakes which can be used in cosmetics. Hydrophilic pigments can include mineral pigments and organic pigments.

Examples of mineral pigments which can be used in the present invention include:

black, yellow, red and brown iron oxides, listed in the Color Index under the references Cl 77499, Cl 77492 and Cl 77491,

manganese violet (Cl 77742),

ultramarine blue (Cl 77007),

ultramarine violet (Cl 77007),

chromium oxide (Cl 77288),

chromium oxide hydrate (Cl 77289) and

ferric blue (Cl 77510),

and mixtures of any of the foregoing.

Examples of organic pigments which can be used in the present invention include the following pigments, optionally on organic lakes:

D & C Red No.3(Cl 45430: 1),

D & C Red No.6(Cl 15850:2),

D&CRedNo.7(Cl 15850: 1),

D & C Red No.9 (Cl 15585:1),

D & C Red No.13 (Cl 15630: 3),

D & C Red No.19 (Cl 45170),

D & C Red No. 21 (Cl 45380: 2),

D & C Red No.27(Cl 45410:1),

D & C Red No. 30 (Cl 73360),

D & C Red No. 36 (Cl 12085),

carbon black (Cl 77266) and lakes based on cochineal carmine (Cl 75470),

D & C Yellow No. 5 (Cl 19140),

D & C Red No. 40 (Cl 16035: 1),

FD & C Blue No. 1 (Cl 42090: 2)

and mixtures of any of the foregoing.

The term “nacres” should be understood as meaning iridescent particles which reflect light.

Examples of nacres which can be used in the present invention include nacreous pigments. Nacreous pigments include mica optionally coated with at least one pigment chosen from organic and mineral pigments, for example titanium oxide and bismuth oxychloride. Nacreous pigments also include titanium mica optionally coated with at least one pigment chosen from organic and mineral pigments, for example iron oxides, ferric blue and chromium oxide. Nacreous pigments may also comprise bismuth oxychloride.

The amount of hydrophilic pulverulent material introduced into the pulverulent mixture generally ranges from 5% to 95% by weight relative to the total weight of the pulverulent mixture, and can also range from 25% to 80% by weight.

In one embodiment, the hydrophilic pulverulent materials can be present in the final composition in a concentration ranging from 5% to 95% by weight relative to the total weight of the final composition, and can also range from 25% to 80% by weight.

In one embodiment, the composition according to the invention can also comprise at least one hydrophobic pulverulent material.

The hydrophobic pulverulent materials can be chosen from pulverulent materials that are hydrophobic by nature, examples include the following:

talc, which is a hydrated magnesium silicate,

hydrophobic polymer powders, for example powders of polyamides for example nylon, for example the product sold under the name “Orgasol 2002 ED NAT COS” by the company Atochem, polyethylene powder, for example the product sold under the name “Coathylene HA 1681” by the company Plast Labor; expanded microspheres made of thermoplastic material, for example the product sold under the name “Expancel 551 DE” by the company Casco-Nobel, polyfluoro powders, for example polytetrafluoroethylene powder, for example the product sold under the name “MP 1400” by the company DuPont de Nemours, silicone powders, for example those sold under the name “Tospearl” by the company Toshiba, acrylic copolymer powders, for example those sold under the name “Polytrap Q5 6603” by the company Dow Chemical, and polystyrene powders for example those sold under the name “Polysphere 3000 SP” by the company Presperese;

lipoamino acids, for example lauroyllysine,

boron nitride,

metal soaps of C₈-C₂₂ carboxylic acids, for example C₁₂-C₁₈ carboxylic acids such as zinc and magnesium stearates, zinc laurate, magnesium myristate,

and mixtures of any of the foregoing.

The hydrophobic pulverulent materials can also be chosen from pulverulent materials that are either hydrophobic in nature or hydrophilic in nature and which have been made hydrophobic by treating them by coating or chemical grafting with products for example silicones, amino acids, metal soaps, fluoro derivatives, mineral oils, lecithin, isopropyl triisostearoyl titanate, polyethylene, collagen and derivatives thereof, and polyacrylates, and mixtures of any of the foregoing.

In one embodiment, the hydrophobic pulverulent materials may be chosen from hydrophobic coated and treated micas, hydrophobic coated and treated silicas, hydrophobic coated and treated kaolin, hydrophobic coated and treated metal oxides for example hydrophobic coated and treated titanium oxides, hydrophobic coated and treated iron oxides and hydrophobic coated and treated zinc oxides, and mixtures of any of the foregoing.

As examples of hydrophobic pulverulent materials, mention may be made of silica microbeads coated with polymethylhydrogenosiloxane, sold under the trade name “Silica SI SB 700” by Miyoshi and sericite coated with methicone/hydrogenated egg oil, sold under the trade name “Sericite SNI S100” by Miyoshi.

The amount of hydrophobic pulverulent material introduced into the pulverulent mixture generally ranges from 5% to 60% by weight relative to the total weight of the pulverulent mixture, and can range from 10% to 45% by weight.

In one embodiment, the hydrophobic pulverulent materials can be present in the final composition at a concentration ranging from 5% to 60% by weight relative to the total weight of the final composition, and can range from 10% to 45% by weight.

The weight ratio of the amount of hydrophilic pulverulent material relative to the amount of hydrophobic material generally ranges from 0.08 to 15, and can range from 0.40 to 8.

In one embodiment, the final composition can comprise at least one pigment, and can also comprise at least two different pigments.

In one embodiment, the pigments can be present at a concentration ranging from 1% to 30% by weight relative to the total weight of the final composition and can also range from 10% to 20% by weight.

In one embodiment, the final composition can comprise at least one nacre.

The nacres are generally present at a concentration ranging from 1% to 50% by weight relative to the total weight of the final composition, and can range from 15% to 25% by weight.

The compositions of the invention can also comprise flakes chosen from polymers and copolymers both chosen from polyethylene/terephthalate and polymethacrylate, which are optionally coated. These flakes may be present in the composition according to the invention at a concentration ranging up to 10% by weight relative to the total weight of the composition.

In one embodiment, the final composition can comprise at least one pulverulent material with a mean particle size of greater than or equal to 100 nanometers. The pulverulent material with a mean particle size of greater than or equal to 100 nanometers can be present in the pulverulent mixture at a concentration ranging from 10% to 50% by weight, and can range from 15% to 35% by weight relative to the weight of the pulverulent mixture.

In one embodiment, the pulverulent material with a mean particle size of greater than or equal to 100 nanometers can be present in the final composition at a concentration ranging from 10% to 50% by weight relative to the total weight of the final composition, and can range from 15% to 35% by weight.

In one embodiment, in order to ensure that the final composition has good cohesion, at least one fatty substance can be added to the pulverulent phase. Examples of fatty substances which may be used, include: mineral oils, for example liquid petroleum jelly; oils of animal origin, for example lanolin; oils of plant origin, for example jojoba oil; esters of carboxylic acids and fatty alcohols, such as C₁₀-C₂₂ fatty alcohols; esters of fatty acids and of alcohol, for example isopropyl palmitate, fatty alcohols, for example fatty alcohols of C₁₀-C₂₂, such as oleyl alcohol, isostearyl alcohol and octyldodecanol; synthetic oils, for example poly-α-olefins; synthetic oils which may be optionally hydrogenated, for instance polyisobutene (parleam), polydecenes; silicone oils, for example phenylsilicone oils, silicone gums and silicone waxes, for example alkyldimethicones; and fluoro oils.

The amount of fatty substance added to the pulverulent mixture generally ranges from 0.1% to 20% by weight relative to the total weight of the pulverulent mixture, and can range from 0.5% to 15% by weight.

In one embodiment, the fatty substances can be present in the final composition at a concentration ranging from 0.1% to 20% by weight relative to the total weight of the final composition, and can also range from 0.5% to 15% by weight.

In one embodiment, the pulverulent mixture can further comprise at least one surfactant to facilitate the wetting and dispersing of the pulverulent mixture. The surfactant can generally be of nonionic nature, for instance polyoxyethylenated sorbitan esters; of cationic nature, for instance quaternary ammonium salts; or of amphoteric nature, for instance betaine derivatives.

The amount of surfactant introduced into the pulverulent mixture can range from 0.1% to 10% by weight relative to the total weight of the pulverulent mixture.

In one embodiment, the surfactant can be present in the final composition at a concentration ranging from 0.1% to 10% by weight relative to the total weight of the final composition.

The aqueous phase of the present invention contains water in an amount which is sufficient to hydrate the plaster to calcium sulphate dihydrate. In one embodiment, it can optionally comprise at least one additive chosen from water-soluble additives and water-dispersible additives. Examples of additives include water-soluble polymers, water-dispersible polymers, surfactants and waxes. Cosmetic active agents may also be added to the aqueous phase, for example, moisturizers for example glycerol, propylene glycol, O antioxidants and sunscreens.

The aqueous phase generally may be mixed with the pulverulent mixture, so as to obtain a castable mixture, in a weight proportion of the pulverulent mixture to the aqueous phase ranging from 0.2 to 2, and can also range from 0.5 to 1.5.

In one embodiment, it is also possible to add to the composition a preserving system which may be introduced into at least one phase chosen from the pulverulent phase and the aqueous phase. This preserving system may be in liquid form and introduced into the composition as described in EP 864 322.

When the paste obtained by mixing the pulverulent mixture and the aqueous phase is cast into a mold, the mixture is left to harden at room temperature. To accelerate the setting time, it is possible to leave the paste to dry by placing the molds in a heated chamber at a temperature which may be, for example, up to 25° C. The composition is then left to dry, for example at a temperature of 55° C. When the final composition is fully dry, it can be used directly in its mold. The composition can also be removed from its mold and placed in suitable packaging.

The composition according to the invention can also comprise any additive commonly used in cosmetics, for example antioxidants, UV screening agents, dyes, fragrances, essential oils, preserving agents, cosmetic active agents, moisturizers, vitamins, sphingolipids and mixtures of any of the foregoing.

These additives may be present in the composition in a proportion of 0% to 15% by weight relative to the total weight of the composition.

Needless to say, a person skilled in the art will take care to select this or these optional additives, and the amount thereof, such that the advantageous properties of the composition according to the invention are not, or are not substantially, adversely affected by the addition envisaged.

The compositions according to the invention can be in the form of a cosmetic product and, for example, in the form of a make-up product such as a blusher, an eyeshadow, a face powder, a rouge, a foundation, a concealer, a make-up product for the body, and a care product for the body, and a care product for the face.

All numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

Concrete and non-limiting examples illustrating the invention will now be given.

### EXAMPLE 1: (INVENTION)

A pulverulent mixture having the formulation below was prepared:

| | | |
|---|---|---|
| Calcium sulphate hemihydrate | 25 | g |
| Hydrophilic powders | 51.1 | g |
| of which pigments (iron oxides with a mean particle size | 9 | g |
| of greater than or equal to 100 nm) | | |
| of which nacres (titanium micas) | 30 | g |
| Hydrophobic powders | 14.97 | g |
| of which talc | 8.1 | g |
| Fatty substance | 8 | g |
| Preserving system | 0.63% | |
| Formaldehyde/4-aminobutyric acid/calcium salt condensate | 0.3 | g |
| as an 11% dispersion in calcium sulphate, sold under the | | |
| trade name “Retardan GK” by Tricosal | | |

72.41 g of water were added to these 100 g of pulverulent mixture and the mixture was then blended until a castable paste was obtained. Sodium citrate may be added either to the pulverulent mixture or to the aqueous phase, if necessary. The paste was then poured into dishes and dried.

The final composition obtained thus has the formulation below:

| | |
|---|---|
| Calcium sulphate dihydrate | 28.33% |
| Sodium citrate | 0.239% |
| Hydrophilic powders | 48.832% |
| of which pigments (iron oxides) | 8.6% |
| of which nacres (titanium micas) | 28.668% |
| Hydrophobic powders | 14.065% |
| of which talc | 7.74% |
| Fatty substance | 7.645% |
| Preserving system | 0.602% |
| Formaldehyde/4-aminobutyric acid/calcium salt condensate | 0.287% |
| as an 11% dispersion in calcium sulphate, sold under the trade | |
| name “Retardan GK” by Lafarge | |

The product obtained after drying is in the form of a cake and can be taken up easily using a powder puff. It does not break and has no holes or spaces. The outer appearance of the composition is smooth.

### EXAMPLE 2: (COMPARATIVE)

A composition identical to that of Example 1 was prepared, but wherein the formaldehyde/4-aminobutyric acid/calcium salt condensate as an 11% dispersion in calcium sulphate was replaced with a conventional setting-time modifier, namely sodium citrate. The composition obtained sticks to the packaging, crumbles and is unacceptable.

### EXAMPLE 3 (INVENTION)

A pulverulent mixture having the formulation below was prepared:

| | | |
|---|---|---|
| Calcium sulphate hemihydrate | 24.95 | g |
| Hydrophilic powders | 45.3 | g |
| of which pigments (ferric blue) | 2.5 | g |
| of which nacres (titanium micas) | 20 | g |
| Hydrophobic powders | 23.07 | g |
| of which talc | 16.2 | g |
| Fatty substance | 6 | g |
| Preserving system | 0.63 | g |
| Calcium tetrahydrogenophosphate monohydrate, sold | 0.05 | g |
| under the trade name “Retardateur RA 01” by Lafarge | | |

88.68 g of water were added to these 100 g of pulverulent mixture and the mixture was then blended until a castable paste was obtained. Sodium citrate may be added either to the pulverulent mixture or to the aqueous phase, if necessary. The paste was then poured into dishes and dried.

The final composition obtained thus has the formulation below:

| | |
|---|---|
| Calcium sulphate dihydrate | 28.282% |
| Sodium citrate | 0.238% |
| Hydrophilic powders | 43.051% |
| of which pigments (ferric blue) | 2.389% |
| of which nacres (titanium micas) | 19.112% |
| Hydrophobic powders | 22.046% |
| of which talc | 15.481% |
| Fatty substance | 5.733% |
| Preserving system | 0.602% |
| Calcium tetrahydrogenophosphate monohydrate, sold under | 0.048% |
| the trade name “Retardateur RA 01” by Lafarge | |

The product obtained after drying is in the form of a cake and can be taken up easily using a powder puff. It does not break and has no holes or spaces. The outer appearance of the composition is smooth.

### EXAMPLE 4: (COMPARATIVE)

A composition identical to that of Example 3 was prepared, but wherein the calcium tetrahydrogenophosphate monohydrate was replaced with a conventional setting-time modifier, namely sodium citrate. The composition obtained sticks to the packaging, crumbles and is unacceptable.

### EXAMPLE 5 (INVENTION)

A pulverulent mixture having the formulation below was prepared:

| | | |
|---|---|---|
| Calcium sulphate hemihydrate | 24.95 | g |
| Hydrophilic powders | 45.3 | g |
| of which pigments (ferric blue) | 2.5 | g |
| of which nacres (titanium micas) | 20 | g |
| Hydrophobic powders | 22.8 | g |
| of which talc | 16.2 | g |
| Fatty substance | 6 | g |
| Preserving system | 0.63 | g |
| Formaldehyde/4-aminobutyric acid/calcium salt condensate | 0.28% | |
| as an 11% dispersion in calcium sulphate, sold under the | | |
| trade name “Retardan GK” by Lafarge | | |
| Starch-coated gypsum | 0.04% | |

88.68 g of water were added to these 100 g of pulverulent mixture and the mixture was then blended until a castable paste was obtained. Sodium citrate may be added either to the pulverulent mixture or to the aqueous phase, if necessary. The paste was then poured into dishes and dried.

The final composition obtained thus has the formulation below:

| | |
|---|---|
| Calcium sulphate dihydrate | 28.282% |
| Sodium citrate | 0.238% |
| Hydrophilic powders | 43.051% |
| of which pigments (ferric blue) | 2.389% |
| of which nacres (titanium micas) | 19.112% |
| Hydrophobic powders | 21.788% |
| of which talc | 15.481% |
| Fatty substance | 5.733% |
| Preserving system | 0.602% |
| Formaldehyde/4-aminobutyric acid/calcium salt condensate | 0.268% |
| as an 11% dispersion in calcium sulphate, sold under the trade | |
| name “Retardan GK” by Lafarge | |
| Starch-coated gypsum | 0.038% |

The product obtained after drying is in the form of a cake and can be taken up easily using a powder puff. It does not break and has no holes or spaces. The outer appearance of the composition is smooth.

### EXAMPLE 6: (COMPARATIVE)

A composition identical to that of Example 5 was prepared, but wherein the formaldehyde/4-aminobutyric acid/calcium salt condensate has an 11% dispersion in calcium sulphate and the starch-coated gypsum were replaced with a conventional setting-time modifier, namely sodium citrate. The composition obtained sticks to the packaging, crumbles and is unacceptable.

What is claimed is:

1. A composition comprising:
(a) calcium sulphate dihydrate;
(b) at least one setting-kinetics modifier chosen from calcium monophosphates, formaldehyde/4-aminobutyric acid/calcium salt condensates and starch-coated gypsums; and
(c) an acceptable medium.

2. A composition according to claim 1, wherein said composition is a cosmetic composition.

3. A composition according to claim 1, wherein said acceptable medium is chosen from cosmetically acceptable media and physiologically acceptable media.

4. A composition according to claim 1, wherein said setting-kinetics modifier comprises a formaldehydel4-aminobutyric acid/calcium salt condensate dispersed in calcium sulphate.

5. A composition according to claim 4, further comprising starch-coated gypsum.

6. A composition according to claim 1, wherein said setting-kinetics modifier comprises a calcium monophosphate.

7. A composition according to claim 1, wherein said setting-kinetics modifier further comprises sodium citrate.

8. A composition according to claim 1, wherein said setting-kinetics modifier is present at a concentration ranging from 0.001% to 5% by weight relative to the total weight of the final composition.

9. A composition according to claim 8, wherein said concentration ranges from 0.01% to 1% by weight relative to the total weight of the final composition.

10. A composition according to claim 9, wherein said concentration ranges from 0.01% to 0.6% by weight relative to the total weight of the final composition.

11. A composition according to claim 1, wherein said calcium sulphate dihydrate is present at a concentration ranging from 10% to 70% by weight relative to the total weight of the final composition.

12. A composition according to claim 11, wherein said concentration ranges from 15% to 35% by weight relative to the weight of the final composition.

13. A composition according to claim 1, comprising at least one hydrophilic pulverulent material.

14. A composition according to claim 13, wherein said at least one hydrophilic pulverulent material is present at a concentration ranging from 5% to 95% by weight relative to the total weight of the final composition.

15. A composition according to claim 14, wherein said concentration ranges from 25% to 80% by weight relative to the total weight of the final composition.

16. A composition according to claim 13, comprising at least one pigment.

17. A composition according to claim 13, comprising at least two different pigments.

18. A composition according to claim 16, wherein said at least one pigment is present at a concentration ranging from 1% to 30% by weight relative to the total weight of the final composition.

19. A composition according to claim 18, wherein said concentration ranges from 10% to 20% by weight relative to the total weight of the final composition.

20. A composition according to claim 13, comprising at least one nacre.

21. A composition according to claim 20, wherein said at least one nacres is present at a concentration ranging from 1% to 50% by weight relative to the total weight of the final composition.

22. A composition according to claim 21, wherein said concentration ranges from 15% to 25% by weight relative to the total weight of the final composition.

23. A composition according to claim 1, comprising at least one hydrophobic pulverulent material.

24. A composition according to claim 23, wherein said at least one hydrophobic pulverulent material is present at a concentration ranging from 5% to 60% by weight relative to the total weight of the final composition.

25. A composition according to claim 24, wherein said concentration ranges from 10% to 45% by weight relative to the total weight of the final composition.

26. A composition according to claim 23, comprising at least one pigment.

27. A composition according to claim 23, comprising at least two different pigments.

28. A composition according to claim 26, wherein said at least one pigment is present at a concentration ranging from 1% to 30% by weight relative to the total weight of the final composition.

29. A composition according to claim 28, wherein said concentration ranges from 10% to 20% by weight relative to the total weight of the final composition.

30. A composition according to claim 23, comprising at least one nacre.

31. A composition according to claim 30, wherein said at least one nacre is present at a concentration ranging from 1% to 50% by weight relative to the total weight of the final composition.

32. A composition according to claim 31, wherein said concentration ranges from 15% to 25% by weight relative to the total weight of the final composition.

33. A composition according to claim 1, comprising at least one hydrophillic pulverulent material and at least one hydrophobic pulverulent material.

34. A composition according to claim 33, wherein the weight ratio of the amount of hydrophilic pulverulent material relative to the amount of hydrophobic material ranges from 0.08 to 15.

35. A composition according to claim 34, wherein the weight ratio of the amount of hydrophilic pulverulent material relative to the amount of hydrophobic material ranges from 0.40 to 8.

36. A composition according to claim 33, comprising at least one pigment.

37. A composition according to claim 33, comprising at least two different pigments.

38. A composition according to claim 36, wherein said at least one pigment is present at a concentration ranging from 1% to 30% by weight relative to the total weight of the final composition.

39. A composition according to claim 38, wherein said concentration ranges from 10% to 20% by weight relative to the total weight of the final composition.

40. A composition according to claim 33, comprising at least one nacre.

41. A composition according to claim 40, wherein said at least one nacre is present at a concentration ranging from 1% to 50% by weight relative to the total weight of the final composition.

42. A composition according to claim 41, wherein said concentration ranges from 15% to 25% by weight relative to the total weight of the final composition.

43. A composition according to claim 1, comprising at least one pulverulent material with a mean particle size of at least 100 nanometers.

44. A composition according to claim 43, wherein said pulverulent material with a mean particle size of at least 100 nanometers is present in the final composition at a concentration ranging from 10% to 50% by weight relative to the total weight of the final composition.

45. A composition according to claim 44, wherein said concentration ranges from 15% to 35% by weight relative to the total weight of the final composition.

46. A composition according to claim 1, further comprising a fatty substance.

47. A composition according to claim 46, wherein said fatty substance is present at a concentration ranging from 0.1% to 20% by weight relative to the total weight of the final composition.

48. A composition according to claim 47, wherein said concentration ranges from 0.5% to 15% by weight relative to the total weight of the final composition.

49. A composition according to claim 1, further comprising a surfactant.

50. A composition according to claim 49, wherein said surfactant is present at a concentration ranging from 0.1% to 10% by weight relative to the total weight of the final composition.

51. A composition according to claim 1, wherein said composition is a solid composition.

52. A composition according to claim 1, wherein said composition is in the form of a make-up product.

53. A composition according to claim 52, wherein said make-up product is chosen from a blusher, an eyeshadow, a face powder, a rouge, a foundation, a concealer, and a make-up product for the body.

54. A composition according to claim 1, wherein said composition is in the form of a care product chosen from a care product for the body and a care product for the face.

55. A process for manufacturing a composition comprising:
a) preparing a pulverulent mixture comprising at least calcium sulphate hemihydrate in powder form;
b) adding an aqueous phase to said pulverulent mixture;
c) blending said pulverulent mixture and said aqueous phase to obtain a castable mixture;
d) pouring said castable mixture into a mold;
e) leaving said castable mixture in said mold to harden by hydrating the calcium sulphate hemihydrate to calcium sulphate dehydrate; and
f) introducing at least one setting-kinetics modifier chosen from calcium monophosphates, formaldehyde/4-aminobutyric acid/calcium salt condensates, and starch-coated gypsums.

56. A process according to claim 55, wherein said composition is a plaster-based cosmetic composition.

57. A process according to claim 55, wherein said composition is a solid composition.

58. A process according to claim 55, wherein said setting-kinetics modifier comprises a formaldehyde/4-aminobutyric acid/calcium salt condensate dispersed in calcium sulphate.

59. A process according to claim 58, wherein said setting-kinetics modifier further comprising starch-coated gypsum.

60. A process according to claim 55, wherein said setting-kinetics modifier comprises a calcium monophosphate.

61. A process according to claim 55, wherein said setting-kinetics modifier further comprises sodium citrate.

62. A process according to claim 55, wherein said setting-kinetics modifier is added to said pulverulent mixture during the preparation of the pulverulent mixture.

63. A process according to claim 55, wherein said setting-kinetics modifier is present in the pulverulent mixture at a concentration ranging from 0.001% to 5% by weight relative to the weight of the pulverulent mixture.

64. A process according to claim 63, wherein said concentration ranges from 0.01% to 1% by weight relative to the weight of the pulverulent mixture.

65. A process according to claim 64, wherein said concentration ranges from 0.01% to 0.6% by weight relative to the weight of the pulverulent mixture.

66. A process according to claim 55, wherein said pulverulent mixture and said aqueous phase are blended in a weight proportion ranging from 0.2 to 2.

67. A process according to claim 66, wherein said weight proportion ranges from 0.5 to 1.5.

68. A composition manufactured by a process comprising
a) preparing a pulverulent mixture comprising at least calcium sulphate hemihydrate in powder form;
b) adding an aqueous phase to said pulverulent mixture;
c) blending said pulverulent mixture and said aqueous phase to obtain a castable mixture;
d) pouring said castable mixture into a mold;
e) leaving said castable mixture in said mold to harden by hydrating the calcium sulphate hemihydrate to calcium sulphate dehydrate; and
f) introducing at least one setting-kinetics modifier chosen from calcium monophosphates, formaldehyde/4-aminobutyric acid/calcium salt condensates, and starch-coated gypsum.

69. A process according to claim 68, wherein said setting-kinetics modifier comprises a formaldehyde/4-aminobutyric acid/calcium salt condensate dispersed in calcium sulphate.

70. A process for manufacturing a composition comprising calcium sulphate dehydrate and having a smooth and uniform surface, wherein said process comprises use of at least one setting-kinetics modifier chosen from calcium monophosphates, formaldehyde/4-aminobutyric acid/calcium salt condensates, and starch-coated gypsums.

71. A process according to claim 70, wherein said composition is a cosmetic composition.

72. A process according to claim 70, wherein said composition further comprises an acceptable medium chosen from cosmetically acceptable media and physiologically acceptable media.

73. A process according to claim 70, wherein said composition is a solid composition.

74. A process according to claim 70, wherein said composition is in the form of a make-up product.

75. A process according to claim 74, wherein said make-up product is chosen from a blusher, an eyeshadow, a face powder, a rouge, a foundation, a concealer, and a make-up product for the body.

76. A process according to claim 70, wherein said composition is in the form of a care product chosen from a care product for the body and a care product for the face.

77. A process according to claim 70, wherein said setting-kinetics modifier comprises a formaldehyde/4-aminobutyric acid/calcium salt condensate dispersed in calcium sulphate.

78. A process according to claim 70 comprising
a) preparing a pulverulent mixture comprising at least calcium sulphate hemihydrate in powder form;
b) adding an aqueous phase to said pulverulent mixture;
c) blending said pulverulent mixture and said aqueous phase to obtain a castable mixture;
d) pouring said castable mixture into a mold;
e) leaving said castable mixture in said mold to harden by hydrating the calcium sulphate hemihydrate to said calcium sulphate dehydrate; and
f) introducing said at least one setting-kinetics modifier.

79. A process for making up the skin comprising applying to the skin at least one composition comprising:
(a) calcium sulphate dehydrate;
(b) at least one setting-kinetics modifier chosen from calcium monophosphates, formaldehyde/4-aminobutyric acid/calcium salt condensates and starch-coated gypsums; and
(c) an acceptable medium.

80. A process according to claim 79, wherein said composition is a cosmetic composition.

81. A process according to claim 79, wherein said process is non-therapeutic.

82. A process according to claim 79, wherein said acceptable medium is chosen from cosmetically acceptable media and physiologically acceptable media.

83. A process according to claim 79, wherein said composition is a solid composition.

84. A process according to claim 79, wherein said composition is in the form of a make-up product.

85. A process according to claim 84, wherein said make-up product is chosen from a blusher, an eyeshadow, a face powder, a rouge, a foundation, a concealer, and a make-up product for the body.

86. A process according to claim 79, in the form of a care product chosen from a care product for the body and a care product for the face.

87. A process according to claim 79, wherein said setting-kinetics modifier comprises a formaldehyde/4-aminobutyric acid/calcium salt condensate dispersed in calcium sulphate.

88. A process according to claim 79, wherein said composition is manufactured by a process comprising:
a) preparing a pulverulent mixture comprising at least calcium sulphate hemihydrate in powder form;
b) adding an aqueous phase to said pulverulent mixture;
c) blending said pulverulent mixture and said aqueous phase to obtain a castable mixture;
d) pouring said castable mixture into a mold;
e) leaving said castable mixture in said mold to harden by hydrating the calcium sulphate hemihydrate to said calcium sulphate dehydrate; and
f) introducing said at least one setting-kinetics modifier.
